(19) United States
(12) Patent Application Publication
(10) Pub. No.: US 2024/0023866 A1
(43) Pub. Date: Jan. 25, 2024

(54) ELECTROCARDIO-SIGNAL COLLECTION METHOD, SYSTEM AND PREPARATION METHOD THEREOF

(71) Applicant: THE SECOND MEDICAL CENTER OF CHINESE PLA GENERAL HOSPITAL, Beijing (CN)

(72) Inventors: Feng Cao, Beijing (CN); Huiquan Wang, Beijing (CN); Ruihua Cao, Beijing (CN); Yabin Wang, Beijing (CN); Jialiang Miao, Beijing (CN)

(21) Appl. No.: 18/266,087

(22) PCT Filed: Jun. 29, 2021
(86) PCT No.: PCT/CN2021/103170

§ 371 Date: Jun. 8, 2023

(30) Foreign Application Priority Data
May 27, 2021 (CN) 202110586314.4

(51) Int. Cl. A61B 5/282; A61B 5/308; A61B 5/26; A61B 5/273; A61B 5/00

(52) U.S. Cl. CPC A61B 5/282; A61B 5/308; A61B 5/26; A61B 5/273; A61B 5/0006; A61B 5/742; A61B 5/7225; A61B 2560/045; A61B 2560/0468; A61B 2560/0406; A61B 2562/046; A61B 2562/125; A61B 2562/164; A61B 2562/227

(57) ABSTRACT

An electrocardio-signal collection system and method, and preparation method of an electrocardio-signal collection system is provided. The system includes: a collection unit configured to collect an analog electrocardio-signal of a human body, where the collection unit includes a flexible device and 10 conductive electrodes (2), and the conductive electrodes (2) are printed on the flexible device; a control unit connected to the conductive electrodes (2) and configured to process and output the analog electrocardio-signal; and a display unit connected to the control unit and configured to receive an output signal from the control unit and display the output signal. The collection unit in the conductive electrodes (2) printed on the flexible device is used as a collection carrier for an electrocardio-signal, and all the conductive electrodes (2) are put in place at once, thereby improving the convenience of electrocardio-signal collection.

### TECHNICAL FIELD

The present disclosure relates to the technical field of full-lead electrocardio-signal collection, and in particular, to an electrocardio-signal collection system, collection method, and preparation method of electrocardio-signal collection system.

### BACKGROUND

12-lead electrocardiogram is an important basis for clinical diagnosis of cardiovascular diseases, and the 12-lead electrode patches need to be placed in accordance with international universal human anatomical positions. During measurement of 12-lead electrocardio-signal, the electrode patches need to be attached by a professional, which is very inconvenient in emergency due to the complex lead lines.

At present, a patent CN111134657A proposes a rapid measurement device for a full-lead electrocardio-signal, which collects a palm-type electrocardio-signal with gloves as the carrier, is capable of rapidly and accurately locating ten electrodes needed for the full-lead electrocardio-signal, and thus has important significance in emergency. However, the collection electrode of the measurement device is a fabric electrode. Although capable of reused, the electrode will be gradually attached with stains such as dust and sweat which are difficult to remove on its surface with the increase of use times, which seriously affects the effect of the electrocardio-signal collection and brings inconvenience in replacement of the electrode. If the collection electrode is replaced with disposable electrode patches, the stretchability of the electrodes will be affected, and it will take a long time to replace the electrodes one by one.

### SUMMARY

In view of this, an objective of some embodiments of the present disclosure is to provide an electrocardio-signal collection system, collection method and preparation method of electrocardio-signal collection system, where the collection unit with the conductive electrodes printed on the flexible device is used as a collection carrier for an electrocardio-signal, and all the conductive electrodes are put in place at once, thereby improving the convenience of electrocardio-signal collection.

To achieve the above objective, the present disclosure provides the following solutions.

An electrocardio-signal collection system includes:
a collection unit configured to collect an analog electrocardio-signal of a human body, which includes a flexible device and 10 conductive electrodes, and the conductive electrodes are printed on the flexible device;
a control unit connected to the conductive electrodes, which is configured to process and output the analog electrocardio-signal;
a display unit connected to the control unit, which is configured to receive an output signal from the control unit and display electrocardio-data.

Optionally, the collection unit also includes lead lines, the lead lines are printed on the flexible device, and the conductive electrodes are connected to the control unit through the lead lines.

Optionally, the control unit includes:
a signal processing module with a reserved female port, which is connected to the lead lines and configured to filter out noise mixed in the analog electrocardio-signal;
an analog-to-digital conversion module connected to the signal processing module, which is configured to convert an analog electrocardio-signal into a digital electrocardio-signal after filtering out noise;
a control module connected to the analog-to-digital conversion module, which is configured to receive, store and transmit the digital electrocardio-signal, and enables a wireless transmission module and the display unit to operate logically;
the wireless transmission module connected to the control module, which is configured to package and transmit the digital electrocardio-signal to cloud after receiving an instruction from the control module, and the display unit accesses the cloud through a network protocol and displays the digital electrocardio-signal at the cloud in real time;
a power supply module connected to the signal processing module, the analog-to-digital conversion module, the control module and the wireless transmission module, respectively, which is configured to provide working voltages and currents to the signal processing module, the analog-to-digital conversion module, the control module and the wireless transmission module.

Optionally, the display unit is a mobile terminal.

Optionally, the flexible device is a disposable medical rubber glove, with a male joint reserved at a bottom of the disposable medical rubber glove. Each of the conductive electrodes is led out to the male joint through corresponding one of the lead lines, and the male joint is connected to the female port.

Optionally, the electrocardio-signal collection system also includes a semicircular arc wrist ring, and the control unit is provided on the semicircular arc wrist ring.

Optionally, the electrocardio-signal collection system also includes a flexible circuit board, on which the control unit is integrated, and the flexible circuit board is placed within the semicircular arc wrist ring

Optionally, the semicircular arc wrist ring is made of a thin steel sheet bent into a ring along a long axis, and the thin steel sheet is wrapped with silica gel; one end of the semicircular arc wrist ring is provided with a bandage with a loop fastener; another end of the semicircular arc wrist ring is provided with a hook fastener, which is adhered to the the above loop fastener; the female port on the signal processing module extends to an outer surface of the semicircular arc wrist ring; when worn, the semicircular arc wrist ring is disposed such that the flexible circuit board is located on a palm side of a hand, and an opening of the semicircular arc wrist ring is located on a back side of the hand.

A preparation method of an electrocardio-signal collection system, which is configured for preparing the collection unit. The preparation method includes:
drawing a template map, which shows the profile of the glove, including an electrode profile, a line profile, and a male joint profile;
preparing a screen mold according to the template map, where a printing surface of the screen mold includes a glove area containing the glove profile, an electrode area containing the electrode profile, a line area containing the line profile, and a male joint area containing the male joint profile;
applying photosensitive glue to the printing surface of the screen mold except the electrode area, the line area, and the male joint area;
fixing a disposable medical rubber glove to the glove area under the printing surface, pouring a conductive material on a non-printing surface of the screen mold, scraping the conductive material through the electrode area, the line area and the male joint area with a manual scraper, such that the conductive material penetrates through a mesh screen onto the disposable medical rubber glove to obtain the conductive electrodes, the lead lines and the male joint; and applying an insulating material to the lead lines.

An electrocardio-signal collection method applied to the electrocardio-signal collection system, the electrocardio-signal collection method includes:
wearing a disposable medical rubber glove printed with the conductive electrodes, the lead lines and the male joint on a right hand palm of a user;
fixing the semicircular arc wrist ring provided with the control unit at a wrist of the user, and inserting the reserved male joint on the glove into the female port of the control unit;
turning on the power of the control unit, so that the conductive electrodes can be in conduction with the control unit;
placing the right hand palm at a designated position on a chest, and displaying a measured electrocardio-signal by a display unit.

According to specific embodiments of the present disclosure, the following technical effects are disclosed.

The present disclosure discloses an electrocardio-signal collection system, collection method and preparation method of electrocardio-signal collection system. The system includes: a collection unit configured to collect an analog electrocardio-signal of a human body, which includes a flexible device and 10 conductive electrodes, and the conductive electrodes are printed on the flexible device. In the present disclosure, the collection unit with the conductive electrodes printed on the flexible device is used as a collection carrier for the electrocardio-signal, the collection carrier is replaced before each measurement, and the conductive electrodes are put in place at once, which avoids the cumbersome operation of replacing the electrode pieces one by one, and improves the convenience of electrocardio-signal collection.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely parts of embodiments of the present disclosure, and other drawings can be derived from these accompanying drawings by ordinary technicians in this field without creative efforts.

FIG. 1 is a structural diagram of the disposable medical rubber glove with conductive electrodes and lead lines according to Embodiment 1 of the present disclosure;

FIG. 2 is a schematic diagram of the hardware circuit soft board according to Embodiment 1 of the present disclosure;

FIG. 3 is a schematic diagram of the semicircular arc wrist ring according to Embodiment 1 of the present disclosure;

FIG. 4 is a rear view of the wrist ring according to Embodiment 1 of the present disclosure;

FIG. 5 is a front view of the wrist ring according to Embodiment 1 of the present disclosure;

FIG. 6 is a flowchart of the preparation method according to Embodiment 2 of the present disclosure;

FIG. 7 is a schematic diagram of the screen printing plate according to Embodiment 2 of the present disclosure;

FIG. 8 is a flowchart of the collection method according to Embodiment 3 of the present disclosure.

### DESCRIPTION OF REFERENCE SIGNS

1—disposable medical rubber glove, 2—conductive electrode, 3—lead line, 4—male joint, 5—female port, 6—signal processing module, 7—analog-to-digital conversion module, 8—control module, 9—wireless transmission module, 91—antenna, 10—power supply module, 101—lithium battery, 11—flexible circuit board, 12—semicircular arc wrist ring, 121—bandage, 122—loop fastener, 123—hook fastener, 13—screen mold, 14—polyester screen, 15—photosensitive glue.

### DETAILED DESCRIPTION OF THE EMBODIMENTS

The technical solutions in the embodiments of the present disclosure will be described below clearly and completely with reference to the accompanying drawings in the embodiments of the present disclosure. Apparently, the described embodiments are merely parts rather than all of the embodiments of the present disclosure. All other embodiments obtained by technicians in the art based on the embodiments of the present disclosure without creative efforts shall fall within the protection scope of the present disclosure.

An objective of some embodiments of the present disclosure is to provide an electrocardio-signal collection system, collection and preparation method of electrocardio-signal collection system, and the collection unit with the conductive electrodes printed on the flexible device is used as a collection carrier of the electrocardio-signal, and all the conductive electrodes are in place at once, thereby improving the convenience of electrocardio-signal collection.

To make the objective, features, and advantages of the present disclosure clearer and more comprehensible, the present disclosure will be further described in detail below in conjunction with the accompanying drawings and specific embodiments.

### Embodiment 1

The present disclosure provides an electrocardio-signal collection system, including a collection unit, a control unit and a display unit.

The collection unit is configured to collect an analog electrocardio-signal of a human body, which includes 10 conductive electrodes 2, lead lines connected to the conductive electrodes 2, a flexible device, and the conductive electrodes 2 and the lead lines 3 are printed on the flexible device.

The control unit is connected to the conductive electrodes 2 through the lead lines 3 and configured to process and output the analog electrocardio-signal.

The display unit is connected to the control unit and configured to receive an output signal from the control unit and display electrocardio-data.

Optionally, the control unit includes a signal processing module 6, an analog-to-digital conversion module 7, a control module 8, a wireless transmission module 9, and a power supply module 10.

The signal processing module 6 is provided with a reserved female port 5, be connected to the lead lines 3, and configured to filter out noise mixed in the analog electrocardio-signal.

The analog-to-digital conversion module 7 is connected to the signal processing module 6, and configured to convert an analog electrocardio-signal after filtering out noise into a digital electrocardio-signal.

The control module 8 is connected to the analog-to-digital conversion module 7, which is configured to receive, store and transmit the digital electrocardio-signal, enabling the wireless transmission module 9 and the display unit to operate logically.

The wireless transmission module 9 is connected to the control module 8, and is configured to package and transmit the digital electrocardio-signal to cloud after receiving an instruction from the control module.

The power supply module 10 connected to the signal processing module 6, the analog-to-digital conversion module 7, the control module 8 and the wireless transmission module 9, respectively, is configured to provide working voltages and currents for each module.

Specifically, voltages supplied by the power supply module 10 to different modules are not entirely the same.

Optionally, the flexible device—a disposable medical rubber glove 1, is fitted with a reserved male joint 4, which is led to the conductive electrodes 2 through corresponding one of the lead lines 3, and the male joint 4 is connected to the female port 5.

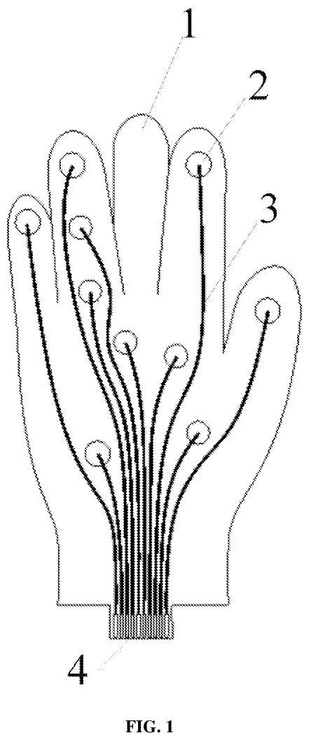

FIG. 1 is a structural diagram of a disposable medical rubber glove printed with conductive electrodes and lead lines according to Embodiment 1 of the present disclosure. In this embodiment, the number of electrodes is 10. As shown in FIG. 1, 10 electrodes are distributed on the disposable medical rubber glove 1, and each electrode has a corresponding lead line 3 which is led out from a center of the electrode and then converged to the bottom of the glove to converge to the reserved male joint 4.

Due to the soft and fragile part of the reserved male joint 4 on the disposable medical rubber glove 1 and in order to facilitate insertion and removal of the reserved male joint 4, a reinforcing material with a thickness of 0.6 mm is attached to the rear of the male joint 4 in this embodiment.

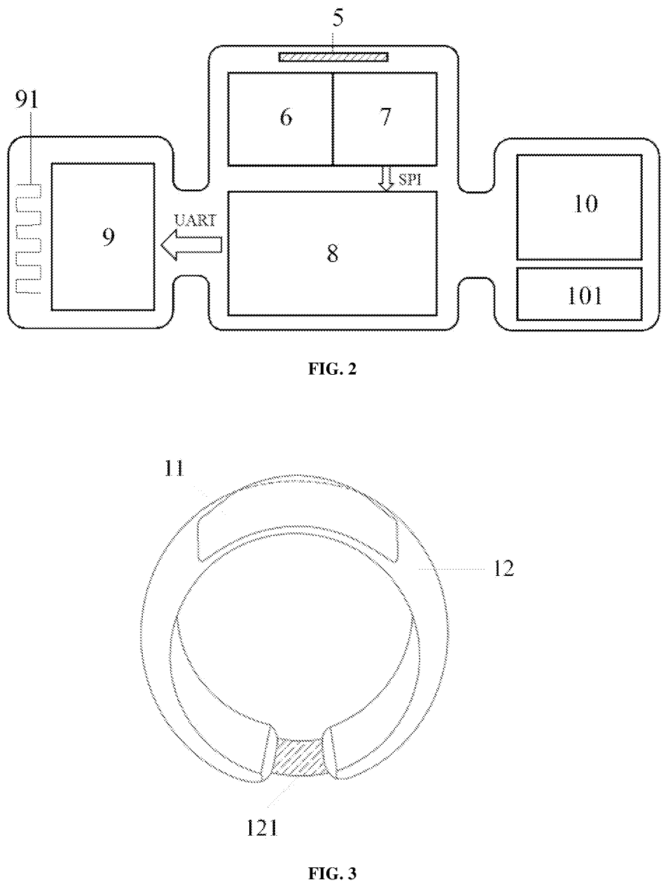

FIG. 2 is a schematic diagram of a hardware circuit soft board according to Embodiment 1 of the present disclosure. As shown in FIG. 2, a reserved female port 5 is provided at the entrance of the signal processing module 6, and the male joint 4 reserved at the bottom of the glove can be inserted into the female port 5 to realize the connection between the front-end analog signal and the back-end circuit. Further, the signal processing module 6 further includes an analog high-pass filter, an analog low-pass filter, and an analog band notch filter. First, the collected analog signal is filtered by the high-pass filter to remove low-frequency noise (such as respiratory wave) and rectify the baseline of the electrocardio-signal. Then, high frequency noise (such as myoelectric wave) is filtered by the low-pass filter to eliminate potential frequency aliasing. Finally, a 50 Hz notch filter is used to eliminate working frequency interference and improve the baseline coarseness.

Specifically, the analog high-pass filter is of Butterworth type, the pass-band cutoff frequency is 100 Hz, the stop-band cutoff frequency is 0.05 Hz, the pass-band maximum attenuation is 1 dB, and the stop-band minimum attenuation is 15 dB.

Alternatively, the analog low-pass filter is of the Butterworth type, the stop-band cutoff frequency is 100 Hz, the pass-band maximum attenuation is 1 dB, and the stop-band minimum attenuation is 15 dB.

As an alternative embodiment, in the analog-to-digital conversion module 7, the peripheral circuit design is mainly carried out around the ADS1298R integrated chip. The chip supports 8 channels of simultaneous sampling with the 24-bit quantization in each channel. Thanks to the high integration and excellent performance of the chip, the size, power and overall cost of the board can be greatly reduced.

In this embodiment, the control module 8 is configured to coordinate the orderly operation of other modules. The control module 8 selects Cortex-M4, which is newly produced by STMicroelectronics (ST) Company, as the core microprocessor controller, and its model is STM32F413VGT6. The analog-to-digital conversion module 7 communicates with the control module 8 through a SPI interface, the control module 8 processes the received data and stores it in the SD card, and the wireless transmission module 9 communicates with the control module 8 through a Universal Asynchronous Receiver/Transmitter (UART) to transmit the processed data.

Specifically, in the wireless transmission module 9, the peripheral circuit design is mainly carried out around the ESP8266 integrated chip. The wireless transmission module 9 can be connected to a designated server through a TCP protocol after being connected to a local area network with an antenna 91, which is convenient to transmit data to a server cloud, and store and summarize the data again in a cloud database.

Alternatively, the power supply module 10 is configured for providing voltage and current to the entire hardware circuit. The power supply module 10 uses a 3.7V lithium battery 101 to supply power to a hardware circuit. When the power is insufficient, the lithium battery 101 can be charged through USB interface. In addition, the power supply module 10 supplies voltage to different modules through different voltage stabilizing chips.

Specifically, the TPS61024DRCR chip provides a voltage of 3V for the control module 8 and the wireless transmission module 9, the LM 27761 chip provides a voltage of −2.5V for the analog-to-digital conversion module 7, and the TLV70025DCKR chip provides a voltage of 2.5V for the analog-to-digital conversion module 7.

Optionally, the display unit visits the cloud through a network protocol and displays the digital electrocardio-signal of the cloud in real time.

In this embodiment, the display unit adopts the mobile terminal for acquiring data from the server in real time and displaying it.

Alternatively, the mobile terminal is a tablet. The tablet is linked to any local area network to access the cloud database through the network protocol and display the synchronization of data on the tablet screen.

Optionally, a semicircular arc wrist ring 12 is also included. The control unit is provided in the semicircular arc wrist ring 12.

The control unit is integrated on a flexible circuit board 11 which is embedded in a semicircular arc wrist ring 12.

Optionally, the flexible circuit board 11 is further included. The control unit is integrated on the flexible circuit board 11, and the flexible circuit board 11 is placed within the semicircular arc wrist ring 12.

Optionally, the semicircular arc wrist ring 12 is made of a thin steel sheet bent into a ring along a long axis, the thin steel sheet is wrapped with silica gel; one end of the semicircular arc wrist ring is provided with a bandage 121, and the bandage 121 is provided with a loop fastener 122; the other end of the semicircular arc wrist ring is provided with a hook and loop fastener 123, and the loop fastener 122 is adhered to the hook fastener 123; the female port 5 on the signal processing module 6 extends to the outer surface of the semicircular arc wrist ring 12; when wearing it, the semicircular arc wrist ring 12 is disposed such that the flexible circuit board 11 is located on a palm side of a hand, and an opening of the semicircular arc wrist ring 12 is located on a back side of the hand.

Specifically, the semicircular arc wrist ring 12 is not worn exactly at the joint where the wrist moves, but lower than the joint, which does not affect the normal movement of the wrist.

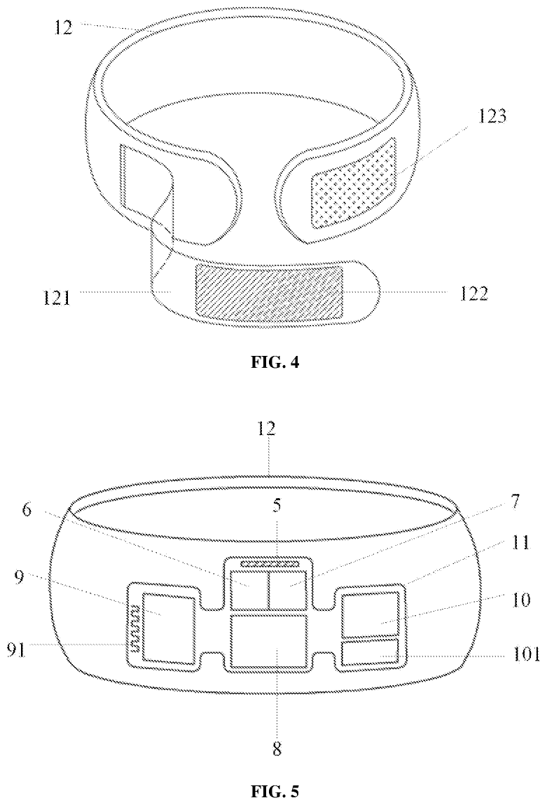

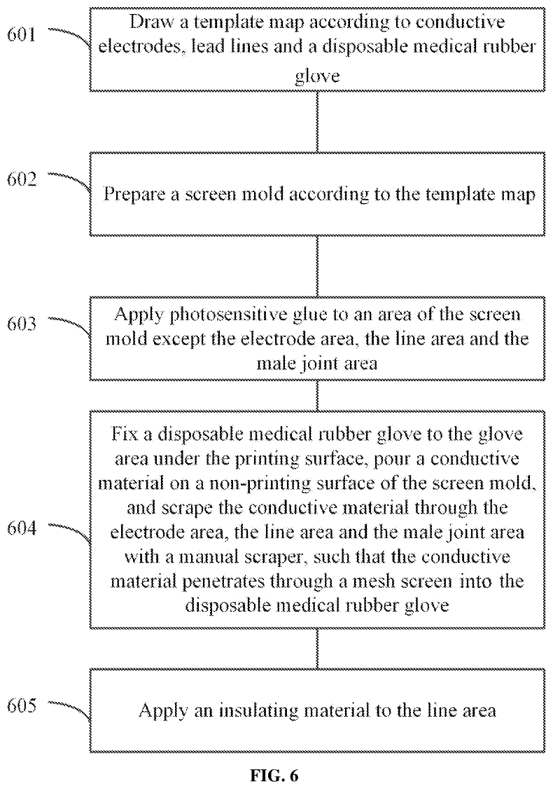

FIG. 3 is a schematic diagram of a semicircular arc wrist ring according to Embodiment 1 of the present disclosure. As shown in FIG. 3, the semicircular arc wrist ring 12 adopts a thin steel sheet with a length of 150 mm and a width of 50 mm. The steel sheet is bent into a ring along a long axis of the steel sheet according to a circular shape with a radius of 40 mm. The wrist ring made of steel sheet is wrapped with silica gel, which protects the users and does not affect flexibility of the wrist ring. FIG. 4 is a rear view of a wrist ring according to Embodiment 1 of the present disclosure. As shown in FIG. 4, one side of the semicircular arc wrist ring 12 is provided with a loop bandage 121, and the other side is provided with a hook fastener 123. The loop bandage 121 can be adhered to the hook fastener 123, which thereby is adaptively fixing the wrist ring to wrists of different people. FIG. 5 is a front view of a wrist ring according to Embodiment 1 of the present disclosure. As shown in FIG. 5, a hardware circuit of an electrocardio-signal collection system is fixed to the wrist ring. The hardware circuit includes the signal processing module 6, the analog-to-digital conversion module 7, the control module 8, the wireless transmission module 9, and the power supply module 10. The hardware circuit is integrated on the FPC flexible circuit board 11 and can be bent along with the wrist ring.

### Embodiment 2

FIG. 6 is a flowchart of a preparation method according to Embodiment 2 of the present disclosure. As shown in FIG. 6, this embodiment provides a preparation method of an electrocardio-signal collection system, including steps 601-605.

In step 601, a template map is drawn according to the conductive electrodes 2, the lead lines 3 and the disposable medical rubber glove 1. The template map includes a glove profile, an electrode profile, a line profile, and a male joint 4 profile.

In this embodiment, a template map is drawn using CAD software. The template map includes an equal proportion profile of the disposable medical rubber glove 1 (medium-sized), an equal proportion profile of the 10 electrodes, an equal proportion line of the lead lines 3, and an equal proportion profile of the reserved male joint 4.

Specifically, the 10 electrodes are uniform in size and circular in shape with a diameter of 10 mm. The reserved male joint 4 consists of 10 rectangles, each of which has a width of 1.3 mm and a length of 10 mm, and the spacing between centers of two adjacent rectangles is 2.54 mm. The line width of the profile of the disposable medical rubber glove 1 is 0.35 mm, the line width of the profile of the electrode is 0.35 mm, the line width of the line of the lead line 3 is 1 mm, and the line width of the profile of the reserved male joint 4 is 0.35 mm.

In step 602, a screen mold 13 is prepared according to the template map. A printing surface of the screen mold 13 includes a glove area containing the glove profile, an electrode area containing the electrode profile, a line area containing the line profile, and a male joint 4 area containing the male joint 4 profile.

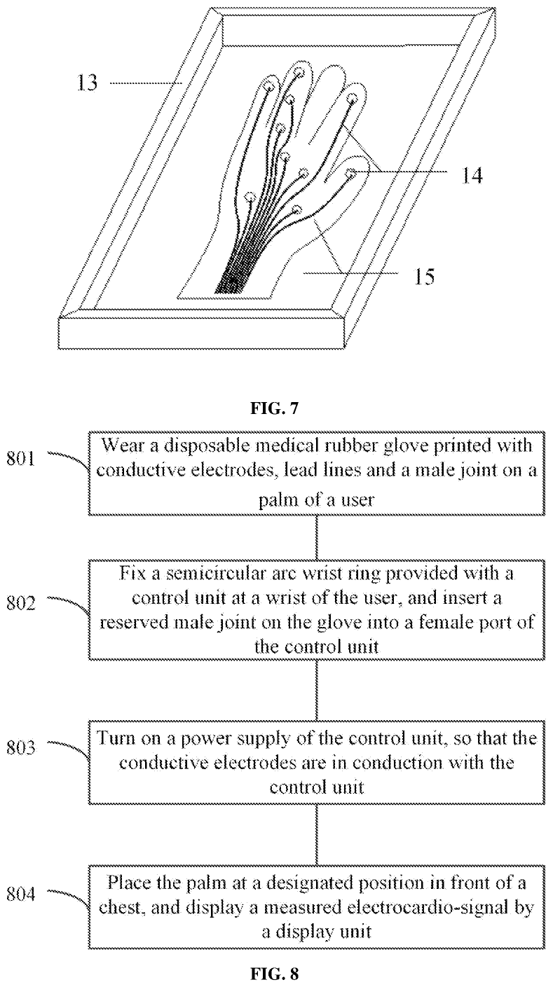

FIG. 7 is a schematic diagram of a screen printing plate according to Embodiment 2 of the present disclosure. As shown in FIG. 7, a bottom surface of the screen mold 13 is a screen printing plate, from which the screen uses a polyester screen 14 with an aperture of 250 mesh.

In step 603, photosensitive glue 15 is applied to an area of the screen mold 13 except the electrode area, the line area and the male joint 4 area.

Specifically, photosensitive glue 1 is applied to all areas except the areas of the electrode profile, the lead line 3 profile and the reserved male joint 4 profile, and the profile of the disposable medical rubber glove 1 is marked on the photosensitive glue 15 using lines with a different color from that of the photosensitive glue 15. The screen area to which the photosensitive glue 15 is applied no longer has a penetration effect, and is translucent.

In step 604, the disposable medical rubber glove 1 is fixed to the glove area under the printing surface, a conductive material is poured on the non-printing surface of the screen mold 13, and the conductive material is scraped through the electrode area, the line area and the male joint 4 area with a manual scraper, such that the conductive material penetrates through a mesh screen onto the disposable medical rubber glove 1.

Specifically, the disposable medical rubber glove 1 is fixed according to the profile of the glove on the photosensitive glue 15, the conductive material is poured on the other side, and the conductive material is scraped through the areas of the electrode, the lead line 3 and the reserved male joint 4 by a manual scraper, so that the electrode material penetrates through a mesh screen onto the rubber glove, thereby completing the printing process. The conductive material is silver paste.

In step 605, an insulating material is applied to the line area to obtain a prepared collection unit.

Specifically, applying the insulating material to the lead line 3 can protect and moisten the lead line 3, to prevent the lead line 3 from the drying and falling off and avoid interference from human sweat during use.

After the prepared collection unit is obtained, the control unit is connected to the collection unit and the display unit respectively to obtain the electrocardio-signal collection system.

### Embodiment 3

FIG. 8 is a flowchart of an collection method according to Embodiment 3 of the present disclosure. As shown in FIG. 8, this embodiment provides an electrocardio-signal collection method, including steps 801-804.

In step 801, a disposable medical rubber glove 1 printed with conductive electrodes 2, lead lines 3 and a male joint 4 is worn on a right hand palm of a user.

Specifically, in the case of non-emergency situation, the adhesive liquid may be applied to the electrode position of the disposable medical rubber glove 1 to make it more firmly fit with skin without adverse effect on signal collection. The solute of the adhesive liquid is a polymeric synthetic polymer.

In step 802, a semicircular arc wrist ring 12 provided with a control unit is fixed to a wrist of the user, and a reserved male joint on the glove is inserted into the female port of the control unit.

Specifically, the semicircular arc wrist ring 12 is worn in such a method that a palm side is upward, the wrist ring is worn on the wrist from top to bottom from the opening, the opening of the wrist ring after wearing is completed should be on the back side of the hand, and the flexible circuit board 11 should be on the palm side.

In step 803, the power supply of the control unit is turned on, so that the conductive electrodes 2 are in conduction with the control unit.

Specifically, press the power button to start the collection system, and the wireless transmission module 9 automatically accesses the local area network. For the first use, it is needed to configure the designated wireless network name for the electrocardio-signal collection system. When used again, the wireless transmission module 9 will automatically access the designated LAN.

In step 804, the palm needs to be placed at a designated position in front of a chest, and then the measured electrocardio-signal is displayed through the display unit.

Specifically, the user can observe the measured full-lead electrocardio-signal on the mobile terminal when holding your right hand open and pressing on the designated positions in front of the chest.

Alternatively, the designated positions are the ten electrode positions mentioned in Patent CN 111134657 A.

The advantageous effects of the present disclosure are as follows.

1. The disposable medical rubber glove can ensure the hygiene of the collection device to the full extent, and can be discarded as a medical waste after use, to avoid cross infection caused by repeated use among different patients.

2. The disposable medical rubber glove is closely attached to the hand, so that the electrodes on the glove has a more natural curve radian when in contact with the skin of a human body, which is beneficial to close contact between the electrodes and the skin.

3. In the present disclosure, a disposable medical rubber glove with conductive electrodes and lead lines is printed by a screen printing plate, and all 10 conductive electrodes are in place for only one time, so as to avoid the cumbersome operation of replacing electrode pieces one by one. The screen printing plate supports batch printing with low production cost and high efficiency.

4. In the present disclosure, the hardware circuit is arranged on a semicircular arc wrist ring, the reserved male joint of the hardware circuit can be flexibly connected with the reserved female port of the disposable medical rubber glove, the replacement of the glove is simple, and the device is portable.

Embodiments of the present specification are described in a progressive way, each embodiment focuses on the difference from other embodiments, and the same and similar parts between the embodiments may refer to each other.

In this specification, some specific embodiments are used for illustration of the principles and implementations of the present disclosure. The description of the foregoing embodiments is used to help illustrate the method of the present disclosure and the core ideas thereof. In addition, those general technical personnel in this field can make various modifications in terms of specific implementations and the scope of application in accordance with the ideas of the present disclosure. In conclusion, the content of this specification shall not be construed as limitations to the present disclosure.

What is claimed is:

1. An electrocardio-signal collection system, comprising:
a collection unit configured to collect an analog electrocardio-signal of a human body, wherein the collection unit comprises a flexible device and 10 conductive electrodes, and the conductive electrodes are printed on the flexible device;
a control unit connected to the conductive electrodes and configured to process and output the analog electrocardio-signal; and
a display unit connected to the control unit and configured to receive an output signal from the control unit and display electrocardio-data.

2. The electrocardio-signal collection system according to claim 1, wherein the collection unit further comprises lead lines, the lead lines are printed on the flexible device, and the conductive electrodes are connected to the control unit through the lead lines.

3. The electrocardio-signal collection system according to claim 2, wherein the control unit comprises:
a signal processing module provided with a reserved female port, connected to the lead lines, and configured to filter out noise mixed in the analog electrocardio-signal;
an analog-to-digital conversion module connected to the signal processing module, and configured to convert an analog electrocardio-signal after filtering out noise into a digital electrocardio-signal;
a control module connected to the analog-to-digital conversion module, and configured to receive, store and transmit the digital electrocardio-signal, and enable a wireless transmission module and the display unit to operate logically;
the wireless transmission module connected to the control module, and configured to package and transmit the digital electrocardio-signal to cloud after receiving an instruction from the control module, wherein the display unit accesses the cloud through a network protocol and displays the digital electrocardio-signal at the cloud in real time;
a power supply module connected to the signal processing module, the analog-to-digital conversion module, the control module and the wireless transmission module, respectively, and configured to provide working voltages and currents to the signal processing module, the analog-to-digital conversion module, the control module and the wireless transmission module.

4. The electrocardio-signal collection system according to claim 3, wherein the display unit is a mobile terminal.

5. The electrocardio-signal collection system according to claim 3, wherein the flexible device is a disposable medical rubber glove, a reserved male joint is provided at a bottom of the disposable medical rubber glove, each of the conductive electrodes is led out to the male joint through corresponding one of the lead lines, and the male joint is connected to the female port.

6. The electrocardio-signal collection system according to claim 5, further comprising a semicircular arc wrist ring, wherein the control unit is provided on the semicircular arc wrist ring.

7. The electrocardio-signal collection system according to claim 6, further comprising a flexible circuit board, wherein the control unit is integrated on the flexible circuit board, and the flexible circuit board is placed within the semicircular arc wrist ring.

8. The electrocardio-signal collection system according to claim 7, wherein the semicircular arc wrist ring is made of a thin steel sheet bent into a ring along a long axis, the thin steel sheet is wrapped with silica gel; one end of the semicircular arc wrist ring is provided with a bandage, the bandage is provided with a loop fastener and the other end of the semicircular arc wrist ring is provided with a hook fastener; the loop fastener is adhered to the hook fastener; the female port on the signal processing module extends to an outer surface of the semicircular arc wrist ring; when worn, the semicircular arc wrist ring is disposed such that the flexible circuit board is located on a palm side of a hand, and an opening of the semicircular arc wrist ring is located on a back side of the hand.

9. A preparation method of an electrocardio-signal collection system, configured for preparing the collection unit according to claim 5, the preparation method comprising:
drawing a template map, wherein the template map comprises a glove profile, an electrode profile, a line profile, and a male joint profile;
preparing a screen mold according to the template map, wherein a printing surface of the screen mold comprises a glove area containing the glove profile, an electrode area containing the electrode profile, a line area containing the line profile, and a male joint area containing the male joint profile;
applying photosensitive glue to the printing surface of the screen mold except the electrode area, the line area, and the male joint area;
fixing a disposable medical rubber glove to the glove area under the printing surface, pouring a conductive material on a non-printing surface of the screen mold, scraping the conductive material through the electrode area, the line area and the male joint area with a manual scraper, such that the conductive material penetrates through a mesh screen into the disposable medical rubber glove to obtain the conductive electrodes, the lead lines and the male joint; and
applying an insulating material to the lead lines.

10. An electrocardio-signal collection method applied to the electrocardio-signal collection system according to claim 8, the electrocardio-signal collection method comprising:
wearing a disposable medical rubber glove printed with the conductive electrodes, the lead lines and the male joint on a right hand palm of a user;
fixing the semicircular arc wrist ring provided with the control unit at a wrist of the user, and inserting the reserved male joint on the glove into the female port of the control unit;
powering on the control unit, so that the conductive electrodes are in conduction with the control unit;
placing the right hand palm at a designated position in front of a chest, and displaying a measured electrocardio-signal by a display unit.
